(19) United States
(12) Patent Application Publication
(10) Pub. No.: US 2024/0374206 A1
(43) Pub. Date: Nov. 14, 2024

(54) Method and System for Generating Notifications Based on Sleep Data

(71) Applicant: Prevayl Innovations Limited, Manchester, Greater Manchester (GB)

(72) Inventor: Rhiannon Jacqueline Goswell, Manchester, Greater Manchester (GB)

(21) Appl. No.: 18/554,848

(22) PCT Filed: Apr. 19, 2022
(86) PCT No.: PCT/GB2022/050974

§ 371 Date: Oct. 11, 2023

(30) Foreign Application Priority Data
Apr. 21, 2021 (GB) 2105698.1

(51) Int. Cl. A61B 5/00; G16H 50/30

(52) U.S. Cl. CPC A61B 5/4809; A61B 5/4812; A61B 5/4815; A61B 5/7264; A61B 5/742; G16H 50/30; A61B 2560/045; A61B 2562/0219

(57) ABSTRACT

A first user electronic device associated with a first user comprises a processor, a memory and a communicator, the memory storing instructions that are executable by the processor (S101). The communicator couples to a sleep monitoring device (S102) and receives sleep data for the first user from the sleep monitoring device (S103). The processor determines, from the sleep data, that a sleep metric exceeds a threshold (S104). The processor generates a notification indicating that the sleep metric exceeds the threshold (S105). The processor controls the communicator to transmit the notification to a second user electronic device associated with a second user (S106). The method enables the sharing of notifications from a first user to a second user based on a sleep metric derived from the sleep data.

The present invention is directed towards a method, user electronic device and system for generating notifications based on sleep data. The present invention is directed, in particular, towards sharing the notifications between a first user electronic device associated with a first user and a second user electronic device associated with a second user.

### BACKGROUND

Sleep monitoring devices, also known as sleep trackers, are becoming increasingly popular. Effectively tracking sleep can be beneficial in identifying an underlying sleep issue for a user so that they can take positive actions to improve their sleep quality in future.

Sleep monitoring devices are provided in a variety of forms. Some sleep monitoring devices may be stand-alone devices positioned in close proximity to the user's bed such as on a bedside table or even under a user's pillow or mattress. Other sleep monitoring devices are provided as or incorporated into a wearable article that may be worn by the user while they are sleeping. Wrist bands are commonly used to incorporate sleep monitoring devices.

Sleep data is measured using a variety of sensors such as acoustic sensors, motion sensors, heart rate sensors and temperature sensors.

International Patent Application Publication No. WO 2016/124817 A1 discloses a family sleep monitoring system. In the system, peripheral devices associated with different family members communicate sleep data to mobile subscriber terminals and further to a data storage application stored on a server computer. The data storage application aggregates the sleep data and displays the sleep data for the family members together to visualize how sleeping behaviour of an individual user in the family influences other users in the same family.

It is an object of the present disclosure to provide an improved mechanism for sharing sleep data amongst users within a family, household, or other group such that positive actions may be provided to improve the sleep quality of the users.

### SUMMARY

According to the present disclosure there is provided a method, user electronic device, and system as set forth in the appended claims. Other features of the invention will be apparent from the dependent claims, and the description which follows.

According to a first aspect of the disclosure, there is provided a method.

The method comprises providing a first user electronic device associated with a first user. The first user electronic device comprises a processor, a memory, and a communicator. The memory stores instructions that are executable by the processor.

The method comprises communicatively coupling, via the communicator, the first user electronic device to a sleep monitoring device arranged to measure sleep data for the first user.

The method comprises receiving, via the communicator, sleep data for the first user from the sleep monitoring device.

The method comprises determining, by the processor from the sleep data, that a sleep metric exceeds a threshold.

The method comprises generating, by the processor, a notification indicating that the sleep metric exceeds the threshold.

The method comprises controlling, by the processor, the communicator to transmit the notification to a second user electronic device associated with a second user.

The method may further comprise providing the sleep monitoring device; measuring, by the sleep monitoring device, the sleep data for the first user; and transmitting, by the sleep monitoring device, the sleep data to the first user electronic device.

The method may further comprise providing the second user electronic device associated with the second user; receiving, by the second user electronic device, the notification from the first user electronic device; and generating, by the second user electronic device, a prompt to the second user based on the notification.

The prompt may indicate that the second user should not disturb the first user.

Generating the prompt may comprise displaying the prompt on a display of the second user electronic device.

The sleep metric may comprise the sleep state of the first user.

The sleep metric may exceed the threshold if the sleep state indicates that the first user was awake beyond a threshold time.

The sleep metric may comprise a sleep disturbance score for the first user.

The sleep-related metric may exceed the threshold if the sleep disturbance score for the first user is greater than a threshold sleep disturbance score.

Controlling the communicator to transmit the notification may comprise controlling the communicator to transmit the notification to a plurality of second user electronic devices associated with different second users.

The notification may be transmitted to the second user electronic device via a server.

The method may further comprise receiving, via a user input unit of the first user electronic device, an input from the first user for triggering the generation of a notification; generating, by the processor, the notification; and controlling, by the processor, the communicator to transmit the notification to the second user electronic device associated with the second user.

According to a second aspect of the disclosure, there is provided a user electronic device associated with a first user.

The user electronic device comprises a processor, a memory, and a communicator. The memory stores instructions that are executable by the processor.

The user electronic device is arranged to be communicatively coupled to a sleep monitoring device via the communicator. The sleep monitoring device is arranged to record sleep-related data for the first user.

The communicator is arranged to receive sleep-related data for the first user from the sleep monitoring device.

The processor is arranged to determine, from the sleep-related data, that a sleep-related metric exceeds a threshold.

The processor is arranged to generate a notification indicating that the sleep-related metric exceeds the threshold.

The processor is arranged to control the communicator to transmit the notification to a second user electronic device associated with a second user.

According to a third aspect of the disclosure, there is provided a system comprising the user electronic device of the second aspect of the disclosure and the sleep monitoring device, wherein the sleep monitoring device is arranged to measure sleep data for the first user and transmit the sleep data to the second user electronic device.

According to a fourth aspect of the disclosure, there is provided a system comprising the user electronic device of the first aspect of the disclosure and the second user electronic device associated with the second user, wherein the second user electronic device is arranged to receive the notification from the user electronic device and generate a prompt to the second user based on the notification.

The sleep monitoring device and the user electronic device are not required to be separate entities in all examples of the present disclosure and instead may be provided as a single device. The single device may be incorporated in a wearable article.

### BRIEF DESCRIPTION OF THE DRAWINGS

Examples of the present disclosure will now be described with reference to the accompanying drawings, in which:

FIG. 1 shows a schematic diagram for an example system according to aspects of the present disclosure;

FIG. 2 shows a schematic diagram of the components of an example user electronic device according to aspects of the present disclosure;

FIG. 3 shows a flow diagram for an example method according to aspects of the present disclosure;

FIG. 4 shows a flow diagram from another example method according to aspects of the present disclosure;

FIG. 5 shows an example user interface according to aspects of the present disclosure;

FIG. 6 shows another example user interface according to aspects of the present disclosure;

FIG. 7 shows a schematic diagram for an example wearable assembly according to aspects of the present disclosure;

FIG. 8 shows a schematic diagram for an example electronics module according to aspects of the present disclosure; and

FIG. 9 shows a schematic diagram for another example electronics module according to aspects of the present disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings but are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

“Wearable article” as referred to throughout the present disclosure may refer to any form of article which may be worn by a user such as a smart watch, necklace, garment, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, garment brassiere, shorts, pants, arm or leg sleeve, vest, jacket/coat, glove, armband, underwear, headband, hat/cap, collar, wristband, stocking, sock, or shoe, athletic clothing, personal protective equipment, including hard hats, swimwear, wetsuit or dry suit.

The term “wearer” includes a user who is wearing, or otherwise holding, the wearable article.

The type of wearable garment may dictate the type of biosignals to be detected. For example, a hat or cap may be used to detect electroencephalogram or magnetoencephalogram signals.

The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the application. Silk may also be used as the natural fibre. Cellulose, wool, hemp and jute are also natural fibres that may be used in the wearable article/garment. Polyester, polycotton, nylon and viscose are synthetic fibres that may be used in the wearable article/garment.

The garment may be a tight-fitting garment. Beneficially, a tight-fitting garment helps ensure that the sensor devices of the garment are held in contact with or in the proximity of a skin surface of the wearer. The garment may be a compression garment. The garment may be an athletic garment such as an elastomeric athletic garment.

The garment has sensing units provided on an inside surface which are held in close proximity to a skin surface of a wearer wearing the garment. This enables the sensing units to measure biosignals for the wearer wearing the garment.

The sensing units may be arranged to measure one or more biosignals of a wearer wearing the garment.

“Biosignal” as referred to throughout the present disclosure may refer to signals from living beings that can be continually measured or monitored. Biosignals may be electrical or non-electrical signals. Signal variations can be time variant or spatially variant.

Sensing components may be used for measuring one or a combination of bioelectrical, bioimpedance, biochemical, biomechanical, bioacoustics, biooptical or biothermal signals of the wearer. The bioelectrical measurements include electrocardiograms (ECG), electrogastrograms (EGG), electroencephalograms (EEG), and electromyography (EMG). The bioimpedance measurements include plethysmography (e.g., for respiration), body composition (e.g., hydration, fat, etc.), and electroimpedance tomography (EIT). The biomagnetic measurements include magnetoneurograms (MNG), magnetoencephalography (MEG), magnetogastrogram (MGG), magnetocardiogram (MCG). The biochemical measurements include glucose/lactose measurements which may be performed using chemical analysis of the wearer's sweat. The biomechanical measurements include blood pressure. The bioacoustics measurements include phonocardiograms (PCG). The biooptical measurements include orthopantomogram (OPG).

The biothermal measurements include skin temperature and core body temperature measurements.

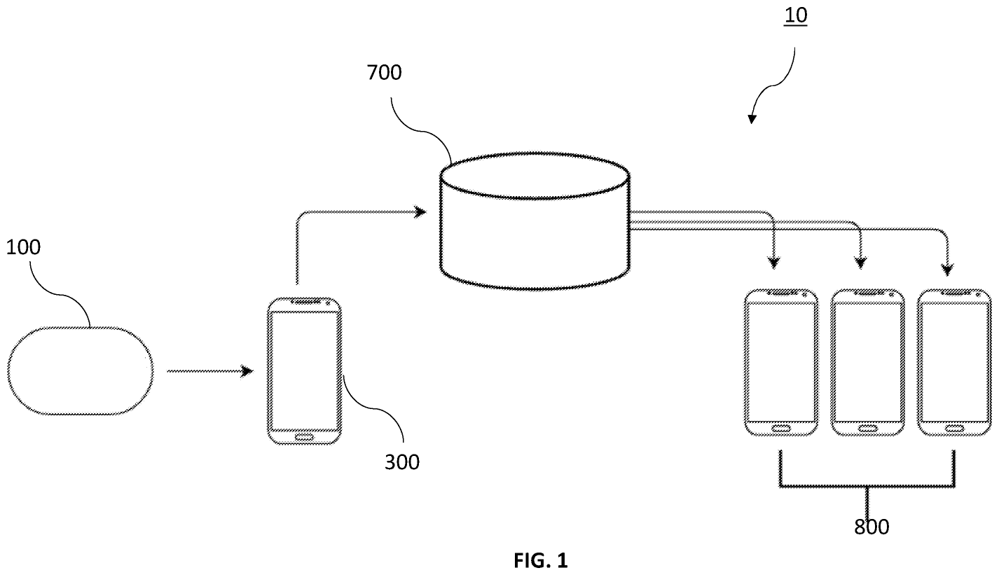

Referring to FIG. 1, there is shown an example system 10 according to aspects of the present disclosure. The system 10 comprises a sleep monitoring device 100, a first user electronic device 300, a server 700 and a plurality of second user electronic devices 800.

The sleep monitoring device 100 is associated with a first user and may be worn by the first user. The sleep monitoring device 100 may be in the form of an electronics module 100 that is able to be (removably) coupled to a wearable article such as a garment worn by the first user.

The sleep monitoring device 100 is communicatively coupled to the first user electronic device 300 over a wireless communication protocol and is able to wireless communicate data such as the sleep data to the first user electronic device 300. Various protocols enable wireless communication between the sleep monitoring device 100 and the first user electronic device 300. Example communication protocols include Bluetooth®, Bluetooth® Low Energy, and near-field communication (NFC).

The first user electronic device 300 is also associated with the first user and may be provided in the vicinity of the first user electronic device 300. The second user electronic devices 800 are associated with second users different to the first user. The second users may be users in the same family/household as the first user, but this is not required in all examples.

The first user electronic device 300 in this example is in the form of a mobile phone or tablet.

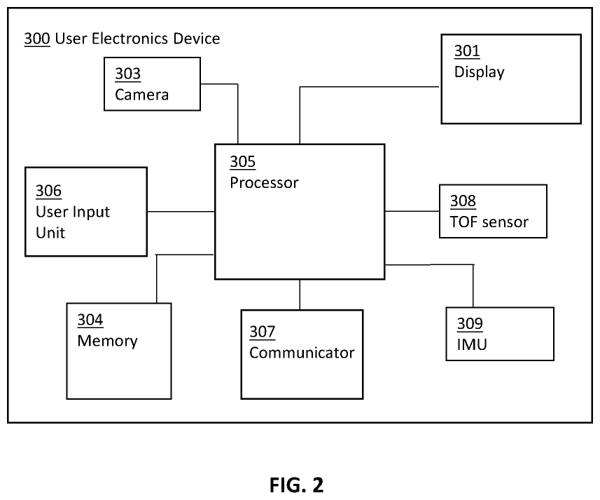

FIG. 2 shows a schematic diagram of the first user electronic device 300. The first user electronic device 300 comprises a processor 305, a memory 304, a wireless communicator 307, a display 301, a user input unit 306, a capturing device in the form of a camera 303 and an inertial measurement unit (IMU) 309. The processor 305 provides overall control to the first user electronic device 300. The processor 305 and memory 304 may be integrally provided together as a controller. The first user electronic device 300 is not required to have all of the elements shown in FIG. 2 and also may include additional elements not shown.

The user input unit 306 receives inputs from the user such as a user credential or commands for controlling the operation of the first user electronic device 300.

The memory 304 stores information for the first user electronic device 300 such as instructions executable by the processor 305.

The display 301 is arranged to display a user interface for applications operable on the first user electronic device 300.

The motion sensor (IMU) 309 provides motion and/or orientation detection and may comprise an accelerometer and optionally one or both of a gyroscope and a magnetometer.

The first user electronic device 300 may also include a biometric sensor. The biometric sensor may be used to identify a user of the device 300 based on unique physiological features. The biometric sensor may be: a fingerprint sensor used to capture an image of the user's fingerprint; an iris scanner or a retina scanner configured to capture an image of a user's iris or retina; an ECG module used to measure the user's ECG; or the camera of the user electronic arranged to capture the face of the user. The biometric sensor may be an internal module of the first user electronic device 300. The biometric module may be an external (stand-alone) device which may be coupled to the first user electronic device 300 by a wired or wireless link.

The processor 305 is configured to launch an application which may be configured to display information such as the sleep data or insights derived from the sleep data received from the sleep monitoring device 100. The sleep data is received from the sleep monitoring device 100 by the wireless communicator 307 of the first user electronic device 300 and input to the processor 305. The application may also enable the first user to control, via the user input unit 306, how their sleep data is shared with the second users operating the second user electronic devices 800.

The display 301 may be a presence-sensitive display and therefore may comprise the user input unit 306. The presence-sensitive display may include a display component and a presence-sensitive input component. The presence sensitive display may be a touch-screen display arranged as part of the user interface.

The first user electronic device 300 is communicatively coupled to the server 700 and is able to transmit and receive data to the server 700 via a wired or wireless communication protocol.

The second user electronic devices 800 are also communicatively coupled to the server 700 and are able to transmit and receive data from the server 700 via a wired or wireless communication protocol. The second user electronic devices 800 may have a similar structure to the first user electronic device 300 shown in FIG. 2.

User electronic devices in accordance with the present invention are not limited to mobile phones or tablets and may take the form of any electronic device which may be used by a user to perform the methods according to aspects of the present invention. The user electronic device may be a smartphone, tablet personal computer (PC), mobile phone, smart phone, video telephone, laptop PC, netbook computer, personal digital assistant (PDA), mobile medical device, camera or wearable device. The user electronic device 300 may include a head-mounted device such as an Augmented Reality, Virtual Reality or Mixed Reality head-mounted device. The user electronic device may be desktop PC, workstations, television apparatus or a projector, e.g. arranged to project a display onto a surface.

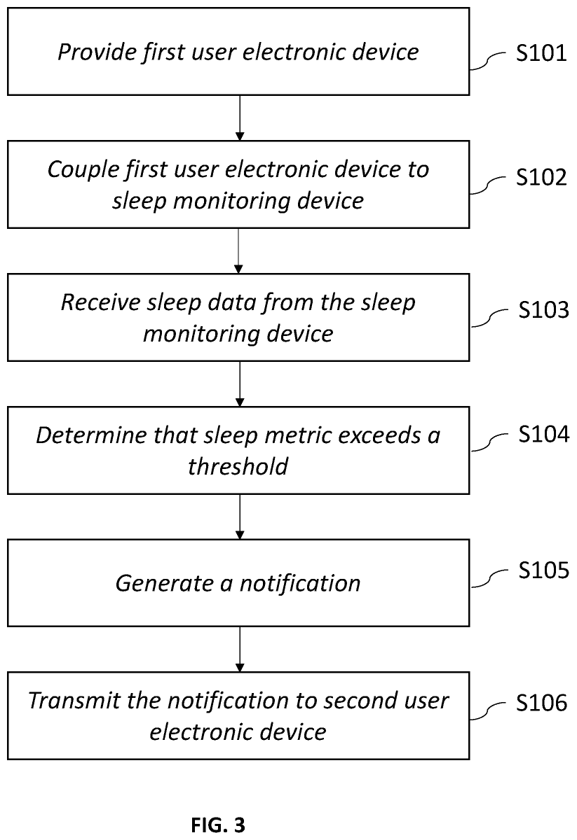

Referring to FIG. 3, there is shown a method performed by the first user electronic device 300 of FIG. 2.

Step S101 comprises providing the first user electronic device 300 associated with the first user. The first user electronic device 300 comprising a processor 305, a memory 304 and a communicator 307, the memory 304 storing instructions that are executable by the processor 305.

Step S102 comprises communicatively coupling, via the communicator 307, the first user electronic device 300 to the sleep monitoring device 100 arranged to measure sleep data for the first user.

The sleep data may include any form of data that indicates the sleep condition of the first user such as whether they are awake, sleeping, moving during sleep, or have a sleep condition such as sleep apnoea. Generally, a user can be identified as being asleep based on their motion, their temperature and/or their heart rate. The sleep data can be determined from one or a combination of these parameters or other parameters such as the galvanic skin response or recorded audio signals.

The one or more sensors may comprise a motion sensor for monitoring the motion of the first user. The motion sensor may comprise an accelerometer, a gyroscope, a magnetometer or a combination of these sensors. The motion sensor may sense motion data which is provided to a processor of the sleep monitoring device 100 or the first user electronic device 300 for determining whether the first user is sleeping. The first user may be determined to be sleeping if they are determined, from the motion data, to be lying down, stationary or generally stationary with small bursts of movements (which may be due to the first user shifting or turning in bed).

The one or more sensors may comprise a temperature sensor such as a skin temperature sensor. The temperature sensor may sense temperature data which is provided to the processor of the sleep monitoring device 100 or the first user electronic device 300 for determining whether the first user is sleeping. The first user may be determined to be sleeping if their skin temperature is lower than normal.

The one or more sensors may comprise a heart rate sensor such as an electrocardiography (ECG) sensor or a pulse sensor. The heart rate sensor may sense heart rate data which is provided to the processor of the sleep monitoring device 100 or the first user electronic device 300 for determining whether the first user is sleeping. The first user may be determined to be sleeping if their heart rate is lower than the typical resting heart rate when the first user is awake. In some examples, the sensing circuitry of the heart rate sensor is part of the sleep monitoring device 100 while the sensing electrodes are provided in a wearable article that the sleep monitoring device 100 is (removably) coupled to.

In addition to determining sleep metrics such as the onset of sleep and the sleep duration, the sleep data measured by the sleep monitoring device can provide other sleep metrics such as information about the sleep quality of the first user. The sleep quality may indicate how disturbed the sleep of the first user is. This may be provided as a sleep disturbance score. The sleep disturbance score may be determined based on parameters such as the number of times the user wakes during their sleep, the movement of the user during sleep, and the proportion of time the user spends in different sleep cycles (e.g. the proportion of REM sleep to non-REM sleep).

Step S103 comprises receiving, via the communicator 307, sleep data for the first user from the sleep monitoring device 100.

Step S104 comprises determining, by the processor 305 from the sleep data, that a sleep metric exceeds a threshold. The sleep metric may be included in the sleep data received from the sleep monitoring device 100. Alternatively, the first user electronic device 300 may process the sleep data to determine the sleep metric.

Step S105 comprises generating, by the processor 305, a notification indicating that the sleep metric exceeds the threshold.

Step S106 comprises controlling, by the processor 305, the communicator 307 to transmit the notification to the second user electronic device 800 associated with a second user.

Advantageously, the present disclosure provides a method for triggering the sharing of notifications from a first user to a second user based on a sleep metric derived from the sleep data. A sleep metric exceeding the threshold, indicates that the first user had a disturbed or delayed sleep. By generating and sharing a notification with the second user electronic device 800, the second user is able to be informed of the poor sleep state of the first user and take appropriate actions such as not attempting to wake the first user, not calling or messaging the first user, and, if they live in the same household, not playing loud music or performing activities such as vacuuming until the first user has woken up.

In addition to sharing the notification, additional information such as the sleep data or the sleep metric may be shared to with the second user. This information may allow the second user to determine when the first user went to sleep or the quality of their sleep (e.g. how often they were disturbed during their sleep).

In some examples, the user electronic device 300 may estimate an optimum wake-up time for the first user based on the sleep data and may send the optimum wake-up time to the second user electronic device 800. The second user electronic device 800 may prompt the second user to wake the first user up at the optimum wake-up time.

The notification may be sent to the second user electronic device 800 immediately or at a predetermined time.

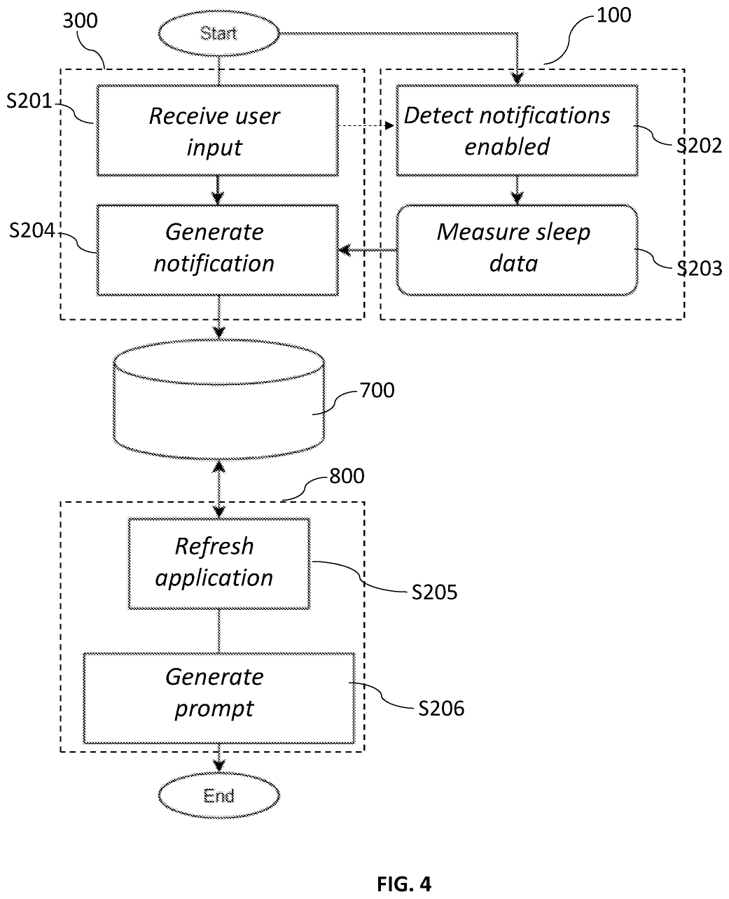

Referring to FIG. 4, there is shown a method performed by the system 10 of FIG. 1.

In the example of FIG. 4, the first user is able to selectively enable whether notifications may be transmitted to the second user electronic device 800. In particular, in step S201, the first user electronic device 300, receives, via the user input unit 306, an input from the first user to enable the generation of notifications.

In some examples, receiving the input from the first user to enable the generation of notifications automatically causes the first user electronic device 300 to generate the notification and transmit the notification to the second user electronic device 800 associated with the second user regardless of the sleep data recording by the sleep monitoring device 100. This enables the first user to cause notifications to be automatically sent if, for example, they know they are going to bed late. This may be beneficial in situations where the first user decides not to wear or use the sleep monitoring device 100 when they go to bed.

If notifications are enabled regardless of the sleep data, the method proceeds to step S204 and steps S202 and S203 are omitted.

In other examples, the input received from the first user is used to enable the first user's sleep data to be used to trigger notifications to the second user. This means that the first user enables their sleep data to be used to share notifications with the second user electronic device 800.

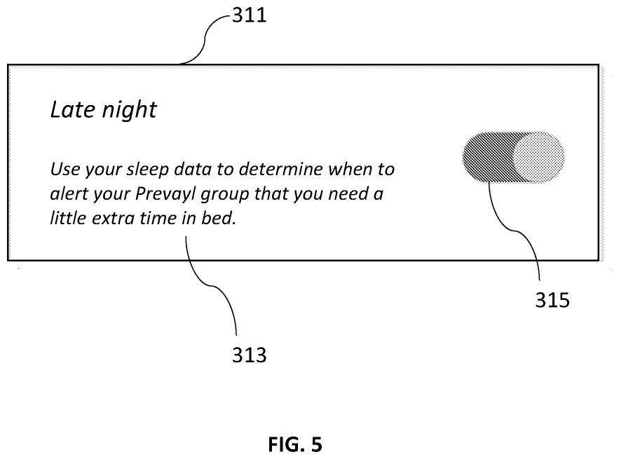

FIG. 5 shows an example user interface 311 that may be displayed to the user on the display 301 of the user electronic device 300 to enable the sending of notifications. The user interface 311 comprises a text box 313 and a toggle switch/checkbox 315 that the first user can interact with to selectively enable the generation of notifications.

In some examples, the user input unit 306 also enables the user to specify the conditions under which a notification is generated. For example, the user can specify the type of sleep metric used to trigger the generation of the notification or adjust the threshold used to trigger the generation of the notification.

The first user may specify that the sleep state is used to trigger the generation of notifications. This may involve the first user specifying the threshold time used to determine if the sleep metric exceeds the threshold. For example, the user may configure the first user electronic device 300 such that notifications are generated if the first user is awake beyond a certain threshold time (e.g. 2 AM).

The first user may use the user input unit 306 to configure when notifications are enabled (e.g. only on the weekends or non-working days).

The first user may use the user input unit 306 to configure which second users the notifications should be sent to.

The user electronic device 300 may forward the user input to the sleep monitoring device 100.

In step S202, the sleep monitoring device 100 determines whether notifications are enabled. This may be based on whether a user input has been received from the user electronic device 300. In other examples, the notifications may be enabled via the sleep monitoring device 100 such as by interacting with a user input unit of the sleep monitoring device 100. Step S202 is not required in all examples as notifications may be always enabled in some examples.

In step S203, the sleep monitoring device 100 monitors sleep data for the first user and transmits the sleep data to the first user electronic device 300. The sleep data may comprise the sleep metric. The sleep monitoring device 100 may perform processing operation on the sleep data to determine the sleep metric. In other examples, the first user electronic device 300 performs the processing to determine the sleep metric.

In step S204, the first user electronic device 300 generates the notification and transmits the notification to the second user electronic device 800 via the server 700.

In examples, where the first user has enabled the automatic generation of notifications, the first user electronic device 300 generates and transmits the notification without considering the sleep data.

In examples where the sleep data is used to determine when to send the notifications, the first user electronic device 300 determines, from the sleep data, whether the sleep metric exceeds the threshold, and if so, generates the notification and transmits the notification.

The server 700 forwards the notification to the second user electronic device 800.

The second user electronic device 800 refreshes an application running on the second user electronic device 800 in step S205 and generates a prompt to the user based on the notification in step S206.

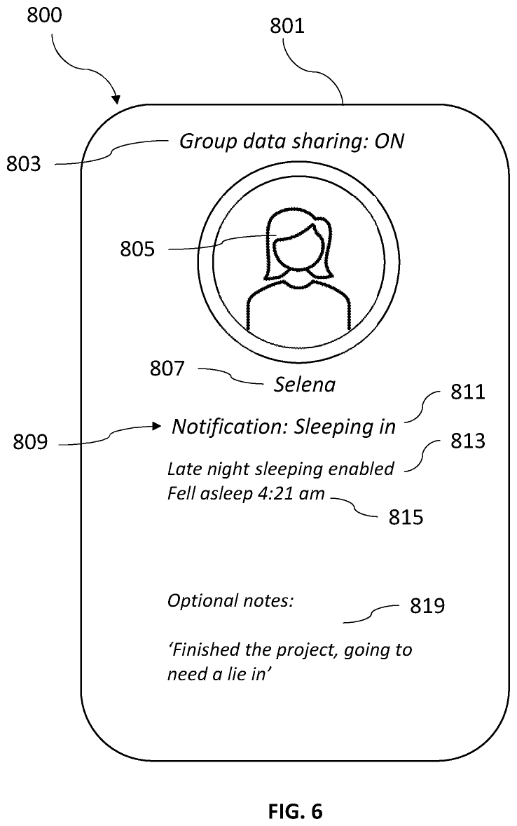

Referring to FIG. 6, there is shown an example user interface 801 displayed on second user electronic device 800. The user interface 801 displays a profile for the first user. The profile includes a picture 805 of the first user along with their name.

A text box 803 is displayed showing whether the sharing of sleep data is enabled for the first user. In this example, the sharing of sleep data is enabled and the text box 803 identifies that group data sharing is on.

The profile further displays the prompt 809 derived from the notification generated by the first user electronic device 300 and transmitted to the second user electronic device 800. The prompt 800 in this example includes a text box 811 informing the second user that the user is sleeping in and thus should not be disturbed. Text box 813 identifies that late night sleeping mode has been enabled for the first user electronic device 300. Late night sleeping mode triggers the generation of the notification if the first user is awake past a threshold time. Text box 815 displays additional information obtained from the sleep data for the first user. The additional information identifies the time the first user fell asleep (4:21 am) in this example.

The profile further includes an optional note 819 that may be input by the first user using the first user electronic device 300 and subsequently transmitted to the second user electronic device 800. The optional note 819 may provide additional context to the second user. In this example, the optional note 819 identifies that the first user has finished their project and needs a lie in. This provides an additional cue to the second user to not disturb the first user.

The present disclosure is not limited to sharing notifications generated from sleep data amongst family members/members of shared household.

In an example, the notification may be transmitted to an employer. This may be particularly useful in industries where employees have strict sleep requirements or where obtaining adequate sleep is required for effective performance of work duties. Such industries include healthcare, aviation, heavy industries and freight transportation (e.g. truck/lorry drivers).

In an example, the notification may be transmitted to a delivery service that is scheduled to deliver an item to the first user. The notification can inform the delivery service that the first user is sleeping/does not want to be disturbed before a certain time (e.g. until after they have achieved adequate sleep) and can instruct the delivery service to leave the item in a safe place or alter their delivery route to deliver the item at a later time. This can provide efficiencies for the delivery service, as it enables them to schedule their routes more efficiently to avoid unfulfilled deliveries.

In an example, the aspects of the present disclosure may be implemented in a health care setting where staff operating on different shift patterns monitor patients. The patients are the “first users” in this example. Shift changes across a day (e.g. between night and day staff) can result in information about a particular patient being lost. This information may relate to the sleep schedule of the patient for example the time they fell asleep or their sleep quality during the night. While one staff member may know in detail the time the patient went to sleep and how many times they woke during the night, it can be challenging to pass all this information onto the next staff member during a shift change. Advantageously, the notification sharing procedure according to the aspects of the present disclosure enable this sleep information to be shared automatically amongst the staff members allowing a consistent standard of clinical care to be applied.

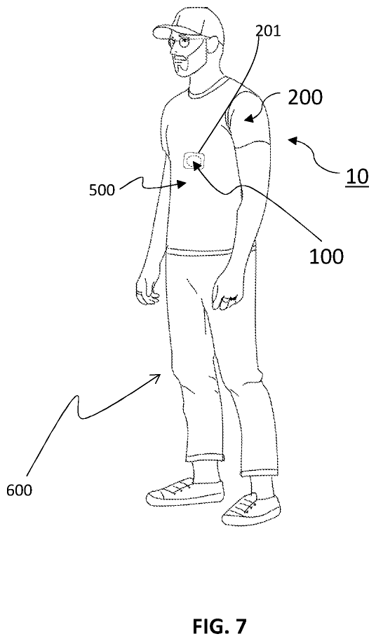

Referring to FIGS. 7-9, there is shown another example wearable assembly 500 comprising the sleep monitoring device 100, and a wearable article 100 in the form of a garment 200. The garment 200 is worn by a user who in this embodiment is the wearer 600 of the garment 200.

The sleep monitoring device 100 is an electronics module 100 that is arranged to integrate with sensing units 400 incorporated into the garment 200 to obtain signals from the sensing units 400. The electronics module 100 and the wearable article 200 and including the sensing units 400 comprise the wearable assembly 500.

The sensing units 400 comprise one or more sensors 209, 211 with associated conductors 203, 207 and other components and circuitry.

The garment 200 has an electronics module holder in the form of a pocket 201. The pocket 201 is sized to receive the electronics module 100. When disposed in the pocket 201, the electronics module 100 is arranged to receive sensor data from the sensing units 400. The electronics module 100 is therefore removable from the garment 200.

The present disclosure is not limited to electronics module holders in the form pockets.

The electronics module 100 may be configured to be releasably mechanically coupled to the garment 200. The mechanical coupling of the electronic module 100 to the garment 200 may be provided by a mechanical interface such as a clip, a plug and socket arrangement, etc. The mechanical coupling or mechanical interface may be configured to maintain the electronic module 100 in a particular orientation with respect to the garment 200 when the electronic module 100 is coupled to the garment 200. This may be beneficial in ensuring that the electronic module 100 is securely held in place with respect to the garment 200 and/or that any electronic coupling of the electronic module 100 and the garment 200 (or a component of the garment 200) can be optimized. The mechanical coupling may be maintained using friction or using a positively engaging mechanism, for example.

Beneficially, the removable electronic module 100 may contain all the components required for data transmission and processing such that the garment 200 only comprises the sensing units 400 e.g. the sensors 209, 211 and communication pathways 203, 207. In this way, manufacture of the garment 200 may be simplified. In addition, it may be easier to clean a garment 200 which has fewer electronic components attached thereto or incorporated therein. Furthermore, the removable electronic module 100 may be easier to maintain and/or troubleshoot than embedded electronics. The electronic module 100 may comprise flexible electronics such as a flexible printed circuit (FPC).

The electronic module 100 may be configured to be electrically coupled to the garment 200.

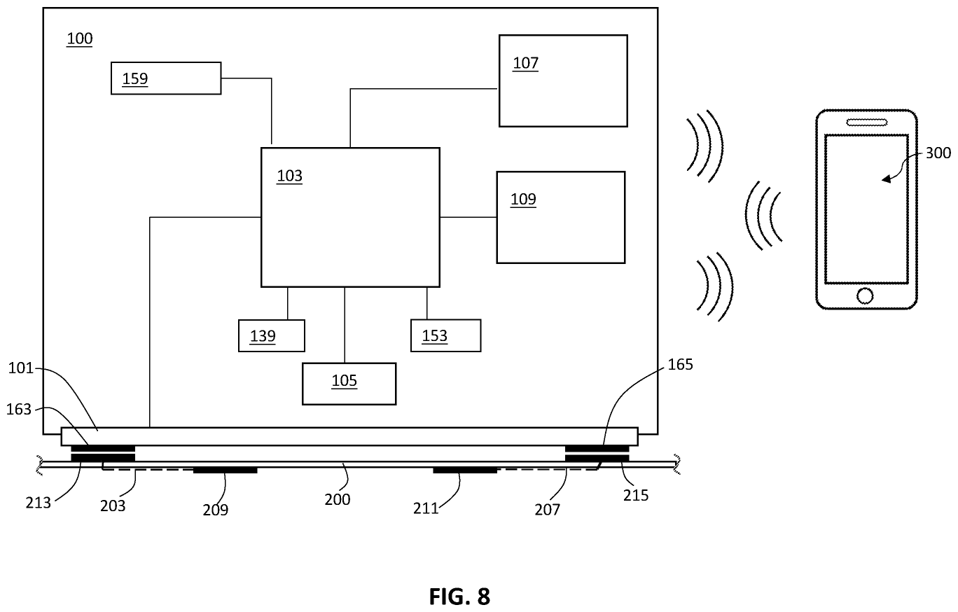

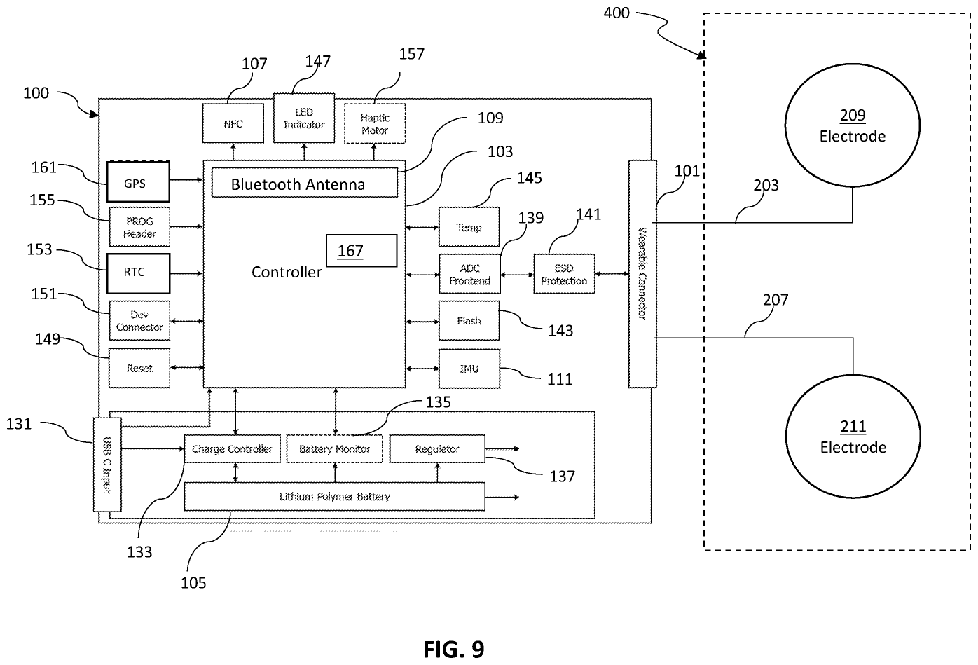

Referring to FIG. 8, there is shown a schematic diagram of an example of the electronics module 100 of FIG. 7. A more detailed block diagram of the electronics components of electronics module 100 and garment are shown in FIG. 9.

The electronics module 100 comprises an interface 101, a controller 103, a power source 105, and one or more communication devices which, in the exemplar embodiment comprises a first antenna 107, a second antenna 109 and a wireless communicator 159. The electronics module 100 also includes an input unit such as a proximity sensor or a motion sensor 111, for example in the form of an inertial measurement unit (IMU).

The electronics module 100 also includes additional peripheral devices that are used to perform specific functions as will be described in further detail herein.

The interface 101 is arranged to communicatively couple with the sensing unit 400 of the garment 200. The sensing unit 400 comprises—in this example—the two sensors 209, 211 coupled to respective first and second electrically conductive pathways 203, 207, each with respective termination points 213, 215. The interface 101 receives signals from the sensors 209, 211. The controller 103 is communicatively coupled to the interface 101 and is arranged to receive the signals from the interface 101 for further processing.

The interface 101 of the embodiment described herein comprises first and second contacts 163, 165 which are arranged to be communicatively coupled to the termination points 213, 215 the respective first and second electrically conductive pathways 203, 207. The coupling between the termination points 213, 215 and the respective first and second contacts 163, 165 may be conductive or a wireless (e.g. inductive) communication coupling.

In this example the sensors 209, 211 are used to measure electropotential signals such as electrocardiogram (ECG) signals, although the sensors 209, 211 could be configured to measure other biosignal types as also discussed above.

In this embodiment, the sensors 209, 211 are configured for so-called dry connection to the wearer's skin to measure ECG signals.

The power source 105 may comprise a plurality of power sources. The power source 105 may be a battery. The battery may be a rechargeable battery. The battery may be a rechargeable battery adapted to be charged wirelessly such as by inductive charging. The power source 105 may comprise an energy harvesting device. The energy harvesting device may be configured to generate electric power signals in response to kinetic events such as kinetic events 10 performed by the wearer 600 of the garment 200. The kinetic event could include walking, running, exercising or respiration of the wearer 600. The energy harvesting material may comprise a piezoelectric material which generates electricity in response to mechanical deformation of the converter. The energy harvesting device may harvest energy from body heat of the wearer 600 of the garment. The energy harvesting device may be a thermoelectric energy harvesting device. The power source 105 may be a super capacitor, or an energy cell.

The first antenna 107 is arranged to communicatively couple with the user electronic device 300 using a first communication protocol. In the example described herein, the first antenna 107 is a passive tag such as a passive Radio Frequency Identification (RFID) tag or Near Field Communication (NFC) tag. These tags comprise a communication module as well as a memory which stores the information, and a radio chip. The user electronic device 300 is powered to induce a magnetic field in an antenna of the user electronic device 300. When the user electronic device 300 is placed in the magnetic field of the communication module antenna 107, the user electronic device 300 induces current in the communication module antenna 107. This induced current triggers the electronics module 100 to retrieve the information from the memory of the tag and transmit the same back to the first user electronic device 300.

The second antenna 109 is arranged to communicatively couple with the first user electronic device 300 over a second wireless communication protocol. The second wireless communication protocol may be a Bluetooth® protocol, Bluetooth® 5 or a Bluetooth® Low Energy protocol but is not limited to any particular communication protocol. In the present embodiment, the second antenna 109 is integrated into controller 103. The second antenna 109 enables communication between the user electronic device 300 and the controller 100 for configuration and set up of the controller 103 and the peripheral devices as may be required. Configuration of the controller 103 and peripheral devices utilises the Bluetooth® protocol.

The wireless communicator 159 may be an alternative, or in addition to, the first and second antennas 107, 109.

Other wireless communication protocols can also be used, such as used for communication over: a wireless wide area network (WWAN), a wireless metro area network (WMAN), a wireless local area network (WLAN), a wireless personal area network (WPAN), Bluetooth® Low Energy, Bluetooth® Mesh, Thread, Zigbee, IEEE 802.15.4, Ant, a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The electronics module 100 includes configured a clock unit in the form of a real time clock (RTC) 153 coupled to the controller 103 and, for example, to be used for data logging, clock building, time stamping, timers, and alarms. As an example, the RTC 153 is driven by a low frequency clock source or crystal operated at 32.768 Hz.

The electronics module 100 also includes a location device 161 such as a GNSS (Global Navigation Satellite System) device which is arranged to provide location and position data for applications as required. In particular, the location device 161 provides geographical location data at least to a nation state level. Any device suitable for providing location, navigation or for tracking the position could be utilised. The GNSS device may include device may include Global Positioning System (GPS), BeiDou Navigation Satellite System (BDS) and the Galileo system devices.

The power source 105 in this example is a lithium polymer battery 105. The battery 105 is rechargeable and charged via a USB C input 131 of the electronics module 100. Of course, the present disclosure is not limited to recharging via USB and instead other forms of charging such as inductive of far field wireless charging are within the scope of the present disclosure. Additional battery management functionality is provided in terms of a charge controller 133, battery monitor 135 and regulator 147. These components may be provided through use of a 30 dedicated power management integrated circuit (PMIC).

The USB C input 131 is also coupled to the controller 131 to enable direct communication with the controller 103 with an external device if required.

The controller 103 is communicatively connected to a battery monitor 135 so that that the controller 103 may obtain information about the state of charge of the battery 105.

The controller 103 has an internal memory 167 and is also communicatively connected to an external memory 143 which in this example is a NAND Flash memory. The memory 143 is used to for the storage of data when no wireless connection is available between the electronics module 100 and a user electronic device 300. The memory 143 may have a storage capacity of at least 1 GB and preferably at least 2 GB.

The electronics module 100 also comprises a temperature sensor 145 and a light emitting diode 147 for conveying status information. The electronic module 100 also comprises conventional electronics components including a power-on-reset generator 149, a development connector 151, the real time clock 153 and a PROG header 155.

Additionally, the electronics module 100 may comprise a haptic feedback unit 157 for providing a haptic (vibrational) feedback to the wearer 600.

The wireless communicator 159 may provide wireless communication capabilities for the garment 200 and enables the garment to communicate via one or more wireless communication protocols to a remote server 700. Wireless communications may include: a wireless wide area network (WWAN), a wireless metro area network (WMAN), a wireless local area network (WLAN), a wireless personal area network (WPAN), Bluetooth® Low Energy, Bluetooth® Mesh, Bluetooth® 5, Thread, Zigbee, IEEE 802.15.4, Ant, a near field communication (NFC), Near Field Magnetic Induction, a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The electronics module 100 may additionally comprise a Universal Integrated Circuit Card (UICC) that enables the garment to access services provided by a mobile network operator (MNO) or virtual mobile network operator (VMNO). The UICC may include at least a read-only memory (ROM) configured to store an MNO or VMNO profile that the garment can utilize to register and interact with an MNO or VMNO. The UICC may be in the form of a Subscriber Identity Module (SIM) card. The electronics module 100 may have a receiving section arranged to receive the SIM card. In other examples, the UICC is embedded directly into a controller of the electronics module 100. That is, the UICC may be an electronic/embedded UICC (eUICC). A eUICC is beneficial as it removes the need to store a number of MNO profiles, i.e. electronic Subscriber Identity Modules (eSIMs). Moreover, eSIMs can be remotely provisioned to garments. The electronics module 100 may comprise a secure element that represents an 35 embedded Universal Integrated Circuit Card (eUICC). In the present disclosure, the electronics module may also be referred to as an electronics device or unit. These terms may be used interchangeably.

The controller 103 is connected to the interface 101 via an analog-to-digital converter (ADC) front end 139 and an electrostatic discharge (ESD) protection circuit 141. The ADC front end 139 may be referred to as a sensing component.

In summary, there is provided a method and system for generating notifications based on sleep data. A first user electronic device associated with a first user comprises a processor, a memory and a communicator, the memory storing instructions that are executable by the processor (S101). The communicator couples to a sleep monitoring device (S102) and receives sleep data for the first user from the sleep monitoring device (S103). The processor determines, from the sleep data, that a sleep metric exceeds a threshold (S104). The processor generates a notification indicating that the sleep metric exceeds the threshold (S105). The processor controls the communicator to transmit the notification to a second user electronic device associated with a second user (S106). The method enables the sharing of notifications from a first user to a second user based on a sleep metric derived from the sleep data.

In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method comprising:
providing a first user electronic device associated with a first user, the first user electronic device comprising a processor, a memory and a communicator, the memory storing instructions that are executable by the processor;
communicatively coupling, via the communicator, the first user electronic device to a sleep monitoring device arranged to measure sleep data for the first user;
receiving, via the communicator, sleep data for the first user from the sleep monitoring device;
determining, by the processor from the sleep data, a sleep metric comprising one or both of a sleep state of the first user and a sleep disturbance score of the first user;
determining by the processor that the sleep metric exceeds a threshold; wherein the sleep metric exceeds the threshold if one or both of the sleep state indicates that the first user was awake beyond a threshold time and the sleep disturbance score for the first user is greater than a threshold sleep disturbance score;
generating, by the processor, a notification indicating that the sleep metric exceeds the threshold;
controlling, by the processor, the communicator to transmit the notification to a second user electronic device associated with a second user;
providing the second user electronic device associated with the second user;
receiving, by the second user electronic device, the notification from the first user electronic device; and
generating, by the second user electronic device, a prompt to the second user based on the notification,
wherein the prompt indicates that the second user should not disturb the first user.

2. The method of claim 1, further comprising:
providing the sleep monitoring device;
measuring, by the sleep monitoring device, the sleep data for the first user; and
transmitting, by the sleep monitoring device, the sleep data to the first user electronic device.

3. The method of claim 1, wherein generating the prompt comprises displaying the prompt on a display of the second user electronic device.

4-7. (canceled)

8. The method of claim 1, wherein controlling the communicator to transmit the notification comprises controlling the communicator to transmit the notification to a plurality of second user electronic devices associated with different second users.

9. The method of claim 1, wherein the notification is transmitted to the second user electronic device via a server.

10. The method of claim 1, further comprising:
receiving, via a user input unit of the first user electronic device, an input from the first user for triggering the generation of a notification;
generating, by the processor, the notification; and
controlling, by the processor, the communicator to transmit the notification to the second user electronic device associated with the second user.

11. A system comprising:
a user electronic device associated with a first user,
the user electronic device comprising a processor, a memory, and a communicator, the memory storing instructions that are executable by the processor,
the user electronic device is arranged to be communicatively coupled to a sleep monitoring device via the communicator, the sleep monitoring device being arranged to record sleep-related data for the first user, wherein
the communicator is arranged to receive sleep-related data for the first user from the sleep monitoring device;
the processor is arranged to determine, from the sleep-related data, a sleep metric comprising one or both of a sleep state of a first user and a sleep disturbance score of the first user; and that the sleep metric exceeds a threshold, wherein the sleep metric exceeds the threshold if one or both of the sleep state indicates that the first user was awake beyond a threshold time and the sleep disturbance score for the first user is greater than a threshold sleep disturbance score;
the processor is arranged to generate a notification indicating that the sleep metric exceeds the threshold; and
the processor is arranged to control the communicator to transmit the notification to a second user electronic device associated with a second user,
the system further comprising a second user electronic device associated with the second user, wherein the second user electronic device is arranged to receive the notification from the user electronic device and generate a prompt to the second user based on the notification.

12. The system of claim 11, further comprising the sleep monitoring device, wherein the sleep monitoring device is arranged to measure sleep data for the first user and transmit the sleep data to the second user electronic device.
